(19) United States
(12) Patent Application Publication
(10) Pub. No.: US 2004/0019339 A1
(43) Pub. Date: Jan. 29, 2004

(54) Absorbent layer attachment

(76) Inventors: Sridhar Ranganathan, Suwanee, GA (US); Dave A. Soerens, Neenah, WI (US); Gabriel H. Adam, Alpharetta, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/206,889

(22) Filed: Jul. 26, 2002

(51) Int. Cl. A61F 13/15; A61F 13/20

(52) U.S. Cl. 604/367; 604/378

(57) ABSTRACT

In an absorbent article, layers or webs of absorbent materials are adhered into a composite to constitute a primary liquid retention layer with improved liquid handling properties. The layers are held together by a discontinuous application of absorbent adhesive in order to improve the fluid intake rate of the composite without sacrifice of overall volume of liquid retention. Control of the viscosity, e.g., concentration of solids, of the liquid absorbent adhesive is taught so as to make its application to, and the drying of, the composite layers practical and efficacious while improving the fluid intake of the absorbent liquid retention layer.

### BACKGROUND OF THE INVENTION

In the field of limited use disposable absorbent articles such as incontinence garments, catamenial products, medical absorbents, and the like, it is very important that the absorbent article be able to acquire the expected volume of liquid at a rate sufficient for the designed purpose and to retain said liquid until the absorbent article is to be disposed of. Thus, the inclusion of various specialization layers in the absorbent article may include layers designed to rapidly take-up and transport, or distribute, liquid to an absorbent core of the article. This absorbent core of the article will then serve to absorb and retain the liquid as the primary liquid retention layer of the garment. Any or all of the various layers of the absorbent article may be a single web or composites of a plurality of webs. Further each layer may serve multiple functions of liquid uptake and retention to some degree.

To this end, the industry has experimented in various degrees with combining webs of material into composites to increase functionality, or combining multiple layers of material to increase functionality, or both, in order to better serve the purposes which the absorbent articles are designed to accomplish. The multiple webs of a single functional layer, or the multiple layers of an article in the process of manufacture, must be held together for at least one of several reasons, including without limitation: processing on the high speed transport lines necessary to accomplish the economical manufacture of the absorbent articles, to provide intimate contact between the webs or layers necessary to accomplish the intended fluid handling functionality of the layers or the article, and to provide visually and tactilely pleasing absorbent articles.

Dual layer absorbents are known in the art. Patents that describe the benefits of dual or multilayered absorbents include U.S. Pat. No. 6,383,960 to Everett et al. Layers of absorbent may be placed together and compressed without an adhesive system or, more typically, the layers are attached with an adhesive. The level of concentration of adhesive, and the wetting properties of the adhesive can play an important role in the layered composites' performance with respect to, among other things, the uptake and retention of liquid. Adhesives are typically hydrophobic and therefore are not friendly for liquid transfer. Further most adhesives are non-absorbent and therefore also serve no liquid retention function.

Some teachings exist in the art which seek to alleviate certain of the above discussed shortcomings. Various teachings in the prior art realize that aqueous solutions of superabsorbent material can be made to adhere to the fibers of a single nonwoven web. For example, patent document EP 0 357 474, to Manning et al., appears to show two cellulosic absorbent layers with a superabsorbent fluid continuously-applied to one of the webs. Manning et al. suggest placing a liquid precursor superabsorbent material on one layer of a two layer composite web and curing to produce the superabsorbent material in situ. However, there is no indication whether Manning et al. intend to adhere the layers together or merely provide one layer with an enhanced absorption capacity.

Absorbent adhesive materials, and especially superabsorbent adhesive materials, in solution form, can be very hard to work with in the practical application of high speed transport lines because the solutions may tend to agglutinize, jamming equipment and making placement imprecise. Also, if too much water is used in an absorbent adhesive solution, the excess water may cause the superabsorbent particles or fibers of the absorbent webs to swell or agglutinate. Furthermore, excess water applied to a cellulose fiber absorbent composite may increase the stiffness of the absorbent. Further, superabsorbent materials that have a glass transition temperature above 40° C. and are applied in a continuous manner, also increase the stiffness of the absorbent structure.

There is a need in the art for methods of adhering webs or layers, or both, in an efficacious and practical manner while retaining or enhancing liquid uptake rates and overall absorbency and product esthetics and flexibility of the concomitant composite webs or layers, or both, which benefit from such improved methods.

### SUMMARY OF THE INVENTION

To solve the above-addressed concerns of the known art, the present invention provides an absorbent composite of two or more webs, or layers, or both, joined by an absorbent adhesive applied between the webs.

At least one of the webs is an absorbent web. The composite absorbent layer is generally designed to be capable of providing the primary liquid retention structure for an absorbent article.

The composite absorbent layer may have a first absorbent web and a second absorbent, uptake, or other, web or layer. The webs or layers can be compressed or densified prior to having a desirably discontinuous layer of adhesive absorbent placed between them to bind the first and second layers. Generally it has been found that a composite absorbent layer according to the present invention, when made with two absorbent airlaid webs, will result in an improved overall fluid intake rate for the composite absorbent structure over that of similar composites joined by a continuous layer of the adhesive absorbent, or similar webs left un-joined, and without sacrifice of overall liquid retention levels. The overall fluid intake rate is judged by a test using three separate fluid applications over a timed period. The present invention especially gives an improved second fluid intake as compared to continuously joined or un-joined webs. The present invention is believed to be applicable to many different varieties, compositions, and types of absorbent webs including cellulosic and non-cellulosic fiber-type webs, and foam-type webs.

The adhesive, according to certain aspects of the present invention, may have between about 2.5% and about 50%, by weight, of an absorbent polymer resin, representing an adhesive precursor material, in a carrier of aqueous liquid, e.g., water or mixtures of water and miscible organic solvents, such that the adhesive may retain sufficient adhesion and absorption while being easily applied by spray equipment to moving webs and easily dried to remove the liquid carrier. The absorbent adhesive precursor material includes water-soluble polymers that are capable of subsequent crosslinking for conversion into absorbent adhesives. A suitable polymer to provide adhesion and absorbent capability along with a low glass transition temperature is poly(ethylene oxide).

The absorbent adhesive precursor materials may be compounds such as ISOBAM® polymer resin (a carboxylated polymer derived from maleic anhydride-isobutylene copolymer) available from Kuraray Co., Ltd. Corporation Japan, or a poly(ethylene oxide) (PEO) polymer resin modified by grafting an alkoxy silane functional group thereto. ISOBAM®-based adhesives can be crosslinked to become absorbent by exposure to heat. PEO-based adhesives will crosslink to become absorbent upon exposure to moisture. Other suitable compounds include copolymers of acrylic acid or methacrylic with hydrophilic copolymers, such as poly(ethylene glycol methacrylate), that provide for a glass transition temperature of the copolymer of less than 25° C. and that incorporate 0.1 to 2.0 mole percent of an acrylate or methacrylate that contains an alkoxy silane functional group capable of providing moisture-induced crosslinking. The particular type of absorbent adhesive will be matched for material compatibility with the web substrates which it is intended to adhere together.

The discontinuous application pattern maybe a plurality of stripes, e.g., a distribution pattern comprising three to five stripes of adhesive per approximately four inches of material wherein the lines are between about one quarter (¼) and about one half (½) inch in width, or about 1 cm in width and about 2.5 cm center to center, although other discontinuous patterns and shapes, whether regular or irregular, may be equally, or more, efficacious within certain morphological and material designations.

Personal care products using this composite are also contemplated to be within the scope of this invention. One such personal care product has a liquid impermeable backsheet, a liquid permeable top sheet, or liner, and a composite absorbent core located between the top sheet and backsheet.

### DEFINITIONS

“Absorbent adhesive precursor material” includes water-soluble polymers that are capable of subsequent crosslinking to convert them into absorbent adhesives. “Adhesive” refers to the absorbent adhesive precursor material in a carrier of aqueous liquid, e.g., water or mixtures of water and miscible organic solvents. As used herein, the term “absorbent adhesive” refers to a material capable of providing bonding between layers such that a separation force of at least 30 grams per centimeter width is required to separate the layers in the dry condition. Furthermore, the absorbent adhesive is capable of absorbing at least 90% by weight of water, under a no load condition.

As used herein an “absorbent web” refers to a material capable of absorbing at least about 5 grams of fluid/grams of dry absorbent material under the most favorable conditions.

“Airlaying” is a well-known process by which a fibrous nonwoven layer can be formed. In the airlaying process, bundles of small fibers having typical lengths ranging from about 1 to about 19 millimeters (mm) are separated and entrained in an air supply and then deposited onto a forming screen, usually with the assistance of a vacuum supply. The randomly deposited fibers then are bonded to one another using, for example, hot air, water compaction, or a spray adhesive. Airlaying is taught in, for example, U.S. Pat. No. 4,640,810 to Laursen et al. Airlaying may include coform deposition which is a known variant wherein pulp or other absorbent fibers or synthetic fibers, or both, are deposited in the same air stream onto the forming screen. The screen may also be referred to herein as a forming wire. “Airformed” refers generically to any nonwoven web forming process not relying on liquid dispersion or distribution of the web.

“Composite” is defined as having two or more discrete components or layers.

“Disposable” includes being disposed of after a single, or limited, use and not intended to be washed and reused.

“Foams” include foam webs made from polyurethane or thermoplastics such as polyethylene, polypropylene, polystyrene or the like, and also includes those foams derived from various processes including extrusion and high internal phase emulsion.

A “layer” is defined as a generally recognizable combination of similar material types or function existing in the X-Y plane.

As used herein, the term “machine direction” means the length of a fabric in the direction in which it is produced. The term “cross direction” or “cross machine direction” means the width of fabric, i.e. a direction generally perpendicular to the machine direction.

“Meltblown fibers” as used herein refers to fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and may be deposited on a collecting surface to form a web of randomly disbursed meltblown fibers. Such a process is disclosed, for example, in U.S. Pat. No. 3,849,241 to Butin et al. Meltblown fibers are microfibers which may be continuous or discontinuous, are generally smaller than 10 microns (μm) in average diameter, and are generally tacky when deposited onto a collecting surface.

As used herein the term “nonwoven fabric or web” means a web having a structure of individual fibers or threads which are interlaid, but not in an identifiable manner as in a knitted fabric. Nonwoven fabrics or webs have been formed from many known processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

“Personal care product” means diapers, wipes, training pants, absorbent underpants, adult incontinence products, feminine hygiene products, wound care items like bandages, and other articles.

“Spunbond fibers” refers to small diameter fibers that are formed by extruding molten thermoplastic material as filaments from a plurality of fine capillaries of a spinneret. Such a process is disclosed in, for example, U.S. Pat. No. 3,802,817 to Matsui et al., U.S. Pat. No. 4,340,563 to Appel et al. The fibers may also have shapes such as those described, for example, in U.S. Pat. No. 5,277,976 to Hogle et al. which describes fibers with unconventional shapes.

“Web” refers to a single construct having major surface dimensions in the x-y plane and a much lesser dimension in the z, or thickness, direction.

Words of degree, such as “about”, “substantially”, and the like are used herein in the sense of “at, or nearly at, when given the manufacturing and material tolerances inherent in the stated circumstances” and are used to prevent the unscrupulous infringer from unfairly taking advantage of the invention disclosure where exact or absolute figures are stated as an aid to understanding the invention.

As used herein and in the claims, the term “comprising” is inclusive or open-ended and does not exclude additional uncredited elements, compositional components, or method steps.

### BRIEF DESCRIPTION OF THE DRAWINGS

The accompanying drawings are presented as an aid to explanation and understanding of various aspects of the present invention only and are not to be taken as limiting the present invention. The drawings are not necessarily to scale, nor should they be taken as photographically accurate depictions of real objects unless otherwise stated.

FIG. 1 is a representative plan view of an absorbent article, specifically an exemplary diaper, laid flat with the interior of the diaper facing the viewer, and with cut-away portions showing various layers of the article.

FIG. 2 illustrates two webs and a discontinuous layer of absorbent adhesive therebetween according to one aspect of the present invention.

FIGS. 3-5 show the absorbent adhesive in alternative discontinuous patterns between two webs.

### DETAILED DESCRIPTION

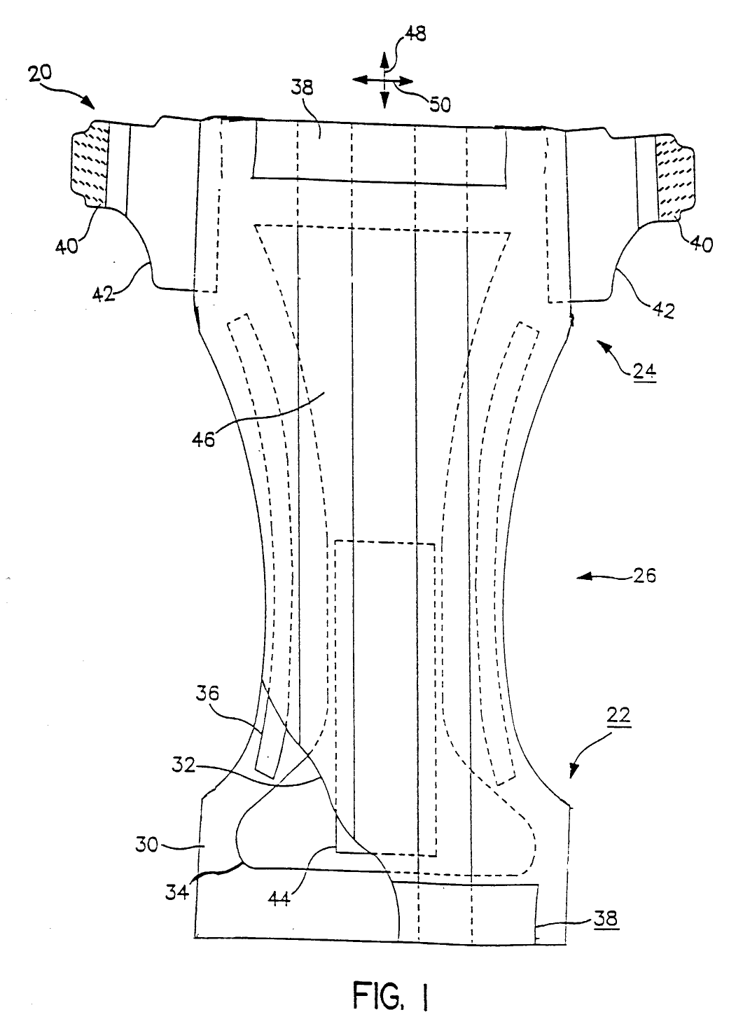

FIG. 1 is a representative plan view of an absorbent article, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs.

The diaper 20 includes, without limitation, an outer cover, or back sheet 30, a liquid permeable bodyside liner, or top sheet, 32 positioned in facing relation with the back sheet 30, and an absorbent core 34, which may be a composite liquid acquisition and retention structure, or a composite liquid retention structure, according to aspects of the present invention as further explained below, and located between the back sheet 30 and the top sheet 32.

The back sheet 30 defines a length, or longitudinal direction 48, and a width, or lateral direction 50 which, in the illustrated embodiment, coincide with the length and width of the diaper 20. The absorbent core 34 generally has a length and width that are less than the length and width of the back sheet 30, respectively. Thus, marginal portions of the diaper 20, such as marginal sections of the back sheet 30, may extend past the terminal edges of the absorbent core 34. In the illustrated embodiments, for example, the back sheet 30 extends outwardly beyond the terminal marginal edges of the absorbent core 34 to form side margins and end margins of the diaper 20. The top sheet 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer.

In the illustrated embodiment, the diaper 20 includes a pair of side panels 42 to which fasteners 40, indicated as the hook portion of a hook and loop fastener, are attached. Generally, the side panels 42 are attached to the side edges of the diaper 20 in one of the waist sections 22, 24 and extend laterally outward therefrom. The side panels 42 may be elasticized or otherwise rendered elastomeric. For example, the side panels 42, or indeed, any precursor component webs of the garment, may be an elastomeric material.

As representatively illustrated in FIG. 1, the disposable diaper 20 may also include a pair of containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

At least certain layers of a composite absorbent core according to this invention, may be made using an airlaid process. The production of airlaid nonwoven composites is well defined in the literature and documented in the art. Examples include the Dan-Web process as described in U.S. Pat. No. 4,640,810 to Laursen et al., the Kroyer process as described in U.S. Pat. No. 4,494,278 to Kroyer et al. and U.S. Pat. No. 5,527,171 to Soerensen; the method of U.S. Pat. No. 4,375,448 to Appel et al.; or other similar methods.

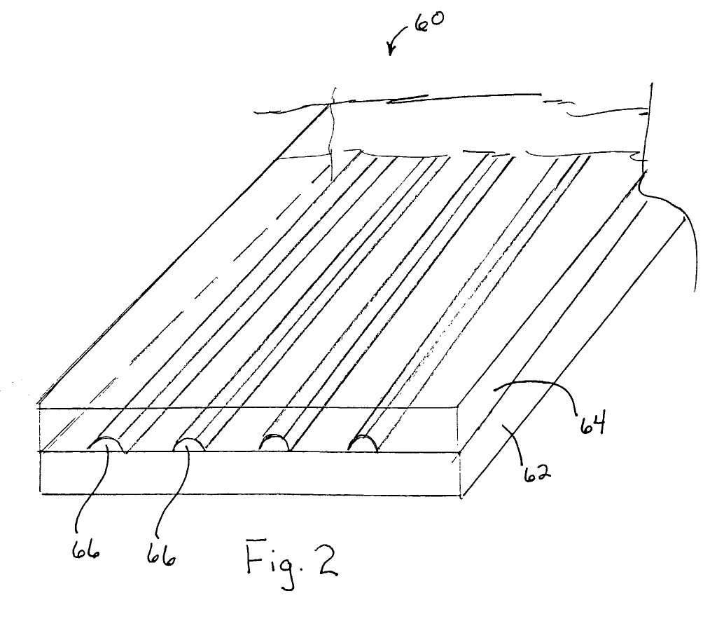

FIG. 2 illustrates a composite 60 of two webs 62, 64 joined by an adhesive 66 according to the present invention. The adhesive 66 is shown as applied in regularly-shaped stripes, although it will be appreciated that such stripes may also include a swirl pattern or the like within the stripes. The first web 62, also sometimes referred to as a substrate, may comprise various types of materials compatible with the adhesive, or its precursors, of the present invention including: cellulosic fiber webs such as airformed webs, thermal bonded airlaid webs, latex bonded airlaid webs and the like, synthetic fiber webs such as spunbond or meltblown webs, carded webs and the like, foams including those made from polyurethane or thermoplastics such as polyethylene, polypropylene, polystyrene or the like, and foams derived from various processes including extrusion and high internal phase emulsion. It is desirable, within the context of the absorbent article of the exemplary embodiment, that at least the substrate layer 62 be an absorbent layer having primary liquid retention functions for the absorbent article. Such a substrate having primary liquid retention functions for the absorbent article may have basis weights of between about 10 gsm to about 1000 gsm. More desirably such a substrate may have basis weights of between about 50 gsm and about 800 gsm, most desirably such a substrate may have basis weights of between about 100 gsm and about 600 gsm.

The second web 64 may be a web of like kind to the first web 62 or may vary in composition or construction so long as the materials thereof are also suitably compatible with the adhesive or absorbent adhesive. The functionality desired from the composite absorbent layer will in large part determine the composition or construction, or both, of the second web. For example, the second web 64 may include the same general types of thermoplastic polymers in its fibers as does the first web 62. But such fibers may be of different morphology, different structural density within the web, have different surfactants, etc. which may give the second web more of a liquid uptake and transport, or so-called “surge”, layer functionality, while the first web retains the primary absorbency and retention functions.

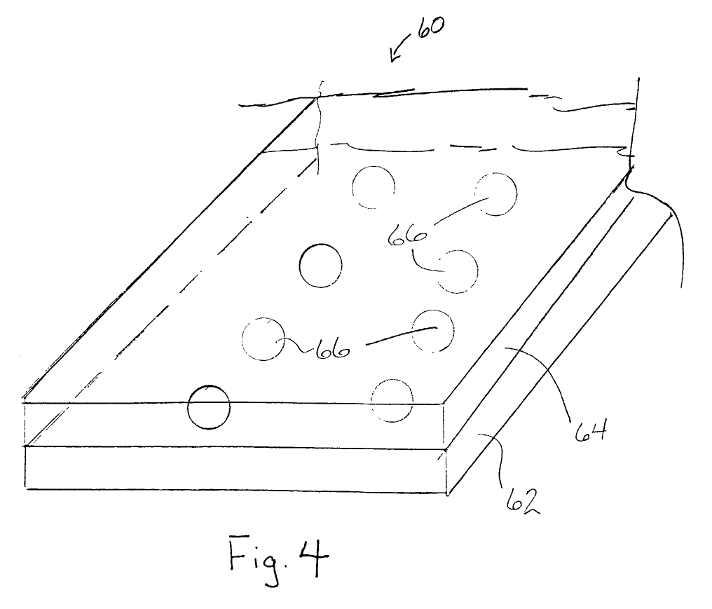

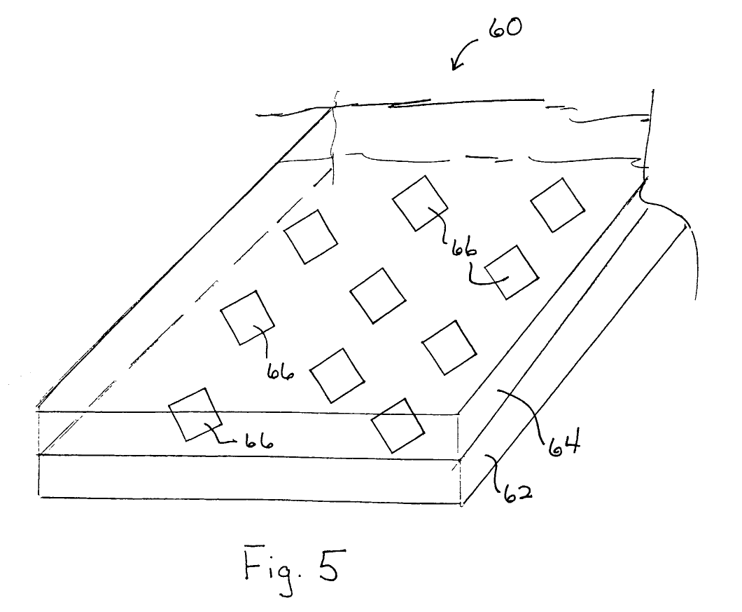

As seen in FIGS. 2-5, the adhesive 66 can be applied in a variety of patterns. Application will typically be done by spray means although other application means may be suitable. The desired pattern for certain aspects of the invention is a discontinuous pattern which facilitates liquid transport throughout the absorbent composite 60. Further, a discontinuous pattern is believed to facilitate a less stiff absorbent structure 34, and of course, uses less overall material thereby making the composite more economical. The adhesive add-on rate for exemplary embodiments of the invention can be between about 1 gsm (grams per square meter) and about 200 gsm of the first and second webs, 62, 64 respectively, more desirably between about 2 gsm and about 150 gsm, and most desirably, between about 5 gsm and about 100 gsm. The adhesive may be applied in a variety of manners and patterns, whether regular or irregular, to the first web 62, second web 64, or both, to achieve a desired patterning of the adhesive 66. Desirably, in the exemplary aspects of the invention, the area of adhesive coverage area is between about 5% and about 95%, more desirably between about 10% and about 80%, and most desirably between about 20% and about 60%.

An adhesive according to one aspect of the invention may include a grafted poly(ethylene oxide) (PEO) that, upon exposure to moisture, crosslinks into a gel structure capable of absorbing relatively large amounts of fluids, such as water or saline. To make an adhesive in accordance with one aspect of the present invention, the absorbent adhesive precursor material, in this case PEO polymer resin, is graft polymerized with an organic moiety containing a trialkoxy silane functional group, or which moiety reacts with water to form a silanol group, hereinafter sometimes referred to collectively as a “silane graft modified PEO”. The silane graft modified PEO polymer resin can be thermally processed into functional forms, such as films, fibers and foams. When these functional forms are exposed to moisture, a crosslinking reaction occurs, by the mechanism shown below, to provide the absorbent adhesive in the form of a gel structure capable of absorbing relatively large amounts of water, such as more than 20 grams of saline per gram of polymer resin under free swell, i.e. no load, conditions, making such materials ideal for use as an absorbent adhesive.

In other aspects of the present invention, water-soluble polymers useful as the absorbent adhesive precursor material include, but are not limited to, poly(alkylene oxides), such as poly(ethylene oxide) (PEO), poly(ethylene glycols), block copolymers of ethylene oxide and propylene oxide, poly(vinyl alcohol) and poly(alkyl vinyl ethers). These water-soluble polymers should be capable of graft polymerization with an organic moiety containing a trialkoxy silane functional group or a moiety that reacts with water to form a silanol group. An exemplary water-soluble polymer for use in this manner is PEO. The absorbent adhesive precursor material PEO resins useful for graft modification in accordance with the present invention include, but are not limited to, PEO resins having initial reported approximate molecular weights ranging from about 10,000 g/mol to about 8,000,000 g/mol as determined by rheological measurements. All molecular weights are given on a weight average basis unless otherwise indicated. Such PEO resins are commercially available from, for example, Union Carbide Corporation having offices in Danbury, Conn., and are sold under the trade designations POLYOX® 205, POLYOX® WSR N-10, POLYOX® WSR N-80, POLYOX® WSRN-750, POLYOX® WSR N-12K and POLYOX® UCARFLOC® Polymer 309. Three particular PEO resins within the above ranges are POLYOX® WSR N-750, POLYOX® WSR N-10 and POLYOX® WSR N-80. These three resins have reported approximate molecular weights, as determined by rheological measurements, of about 100,000 g/mol to 300,000 g/mol.

Organic monomers capable of graft polymerization with PEO, which monomers contain a trialkoxy silane functional group or a moiety that reacts with water to form a silanol group, are useful in the practice of this invention. The trialkoxy silane functional group has the following structure:

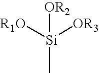

wherein R₁, R₂ and R₃ are alkyl groups independently having from 1 to 6 carbon atoms. The term “monomer(s)” as used herein includes monomers, oligomers, polymers, mixtures of monomers, oligomers and/or polymers, and any other reactive chemical species which is capable of covalent bonding with the parent polymer, PEO.

Ethylenically unsaturated monomers containing a trialkoxy silane functional group are appropriate for this invention and are desired. Desired ethylenically unsaturated monomers include acrylates and methacrylates. A particularly desirable ethylenically unsaturated monomer containing a trialkoxy silane functional group is methacryloxypropyl trimethoxy silane. Methacryloxypropyl trimethoxy silane is commercially available from Dow Corning, having offices in Midland, Mich., under the trade designation Z-6030 Silane.

Other suitable ethylenically unsaturated monomers containing a trialkoxy silane functional group include, but are not limited to, methacryloxyethyl trimethoxy silane, methacryloxypropyl triethoxy silane, methacryloxypropyl tripropoxy silane, acryloxypropylmethyl dimethoxy silane, 3-acryloxypropyl trimethoxy silane, 3-methacryloxypropylmethyl diethoxy silane, 3-methacryloxypropylmethyl dimethoxy silane, and 3-methacryloxypropyl tris(methoxyethoxy) silane. However, it is contemplated that a wide range of vinyl and acrylic monomers having trialkoxy silane functional groups or a moiety that reacts easily with water to form a silanol group, such as a chlorosilane or an acetoxysilane, provide the desired effects to PEO and are effective monomers for grafting in accordance with the present invention.

The amount of organic monomer having trialkoxy silane functional groups or silanol-forming functional groups relative to the amount of PEO may range from about 0.1 to about 20 weight percent of monomer to the weight of PEO. Desirably, the amount of monomer should exceed 0.1 weight percent in order sufficiently to improve the processability of the PEO. A range of grafting levels is demonstrated in the below examples. Typically, the monomer addition levels are between about 1.0% and about 15% of the weight of the base PEO resin; particularly, between about 1.0% and about 10% of the weight of the base PEO resin; especially, between about 1.5% and about 5.5% of the weight of the base PEO resin for some intended uses. A variety of initiators may be useful in the practice of this invention. When grafting is achieved by the application of heat, as in a reactive-extrusion process, it is desirable that the initiator generates free radicals through the application of heat. Such initiators are generally referred to as thermal initiators. For the initiator to function as a useful source of radicals for grafting, the initiator should be commercially and readily available, stable at ambient or refrigerated conditions, and generate radicals at reactive-extrusion temperatures. Compounds containing an O—O, S—S, or N═N bond may be used as thermal initiators. Compounds containing O—O bonds; i.e., peroxides, are commonly used as initiators for graft polymerization. Such commonly used peroxide initiators include: alkyl, dialkyl, diaryl and arylalkyl peroxides such as cumyl peroxide, t-butyl peroxide, di-t-butyl peroxide, dicumyl peroxide, cumyl butyl peroxide, 1,1-di-t-butyl peroxy-3,5,5-trimethylcyclohexane, 2,5-dimethyl-2,5-di(t-butylperoxy)hexane,2,5-dimethyl-2,5-bis(t-butylperoxy)hexyne-3 and bis(a-t-butyl peroxyisopropylbenzene); acyl peroxides such as acetyl peroxides and benzoyl peroxides; hydroperoxides such as cumyl hydroperoxide, t-butyl hydroperoxide, p-methane hydroperoxide, pinane hydroperoxide and cumene hydroperoxide; peresters or peroxyesters such as t-butyl peroxypivalate, t-butyl peroctoate, t-butyl perbenzoate, 2,5-dimethylhexyl-2,5-di(perbenzoate) and t-butyl di(perphthalate); alkylsulfonyl peroxides; dialkyl peroxymonocarbonates; dialkyl peroxydicarbonates; diperoxyketals; ketone peroxides such as cyclohexanone peroxide and methyl ethyl ketone peroxide. Additionally, azo compounds such as 2,2′-azobisisobutyronitrile abbreviated as AIBN, 2,2′-azobis(2,4-dimethylpentanenitrile) and 1,1′-azobis(cyclohexanecarbonitrile) may be used as the initiator.

One aspect of the present invention is demonstrated in the following examples by the use of a liquid, organic peroxide initiator available from R.T. Vanderbilt Company, Inc. of Norwalk, Conn., sold under the trade designation VAROX DBPH peroxide which is a free radical initiator and comprises 2,5-bis(tert butylperoxy)-2,5-dimethyl hexane along with smaller amounts of di(tert butylperoxide). Other initiators may also be used, such as LUPERSOL® 101 and LUPERSOL® 130 available from Elf Atochem North America, Inc. of Philadelphia, Pa.

A variety of reaction vessels may be useful in the practice of this invention. The modification of the PEO can be performed in any vessel as long as the necessary mixing of PEO, the monomer and the initiator is achieved and enough thermal energy is provided to affect grafting. Desirably, such vessels include any suitable mixing device, such as Brabender Plasticorders, Haake extruders, Bandbury mixers, single or multiple screw extruders, or any other mechanical mixing devices which can be used to mix, compound, process or fabricate polymers. In a desired embodiment, the reaction device is a counter-rotating twin-screw extruder, such as a Haake extruder available from Haake, 53 West Century Road, Paramus, N.J. 07652 or a co-rotating, twin-screw extruder, such as a ZSK-30 twin-screw, compounding extruder manufactured by Werner & Pfleiderer Corporation of Ramsey, N.J. It should be noted that a variety of extruders may be used to modify the PEO in accordance with the invention provided that mixing and heating occur.

### EXAMPLE 1

POLYOX WSR N-80 PEO resin was grafted as described above with 6 weight percent of trimethoxy propyl silyl methacrylate. The resin particles were dissolved in water with the aid of a high-speed homogenizer, UltraTurrax model T25, distributed by IKA Work Inc., Willmington, N.C.

A 3% solution, by weight of the above resin was sprayed with air pressurized nozzles onto the surface of an air-laid cellulosic web. Immediately after the solution application a second web of air-laid cellulosic material was joined to the first web by passing the webs through a roll nip and then over a drying can to remove the moisture. The resulting laminate was securely bonded such that attempts to separate the plies resulted in destruction of the sample.

### EXAMPLE 2

Alternatively, the resin solution above was combined with ethylene vinyl acetate latex, Airflex 105, manufactured by Air Products. The ratio of modified POLYOX WSR N-80 to Airflex 105 was 4:1, on a polymer solids basis. The addition of the Airflex 105 improved the spray uniformity and also provided high adhesion between plies. Similar results were obtained with a ratio of modified POLYOX WSR N-80 to Airflex 105 of 2:1.

### EXAMPLE 3

Alternative absorbent adhesives are those which may provide attachment of the layers in either, or both, of a dry state or a wet state, i.e., a solid or semisolid formable, state; or a liquid or sprayable state; respectively, with either state having the possibility of application to substrate webs as a printable material. Such absorbent adhesives will provide very flexible coatings and should therefore have a glass transition temperature below 10° C. and have a bending modulus lower than the webs to which they are applied so that the adhesive will not increase the stiffness of the coated substrate. The absorbent adhesives may include hydrophilic polymers or blends of hydrophilic polymers or hydrophobic polymers containing hydrophilic agents. A particularly preferred adhesive includes acrylic acid copolymers that include, in addition to acrylic acid, long chain, hydrophilic acrylates or methacrylate esters such as poly(ethylene glycol) methacrylate with 1 to 12 ethylene glycol units. In addition, the copolymer includes an acrylate or methacrylate ester that contains an alkoxysilane functionality or a group that is capable, upon exposure to water, of forming a silanol functional group which condenses to form a crosslinked polymer.

Such a copolymer can be prepared as follows.

An initiator solution is prepared by dissolving an initiator in a solvent which will not affect the particle properties. Possible solvents include, but are not limited to, alcohols with ethanol as a more preferred solvent. The polymerization initiator may be activated using a variety of methods including, but not limited to, thermal energy, ultraviolet light, or redox chemical reactions. A preferred class of initiators are organic peroxides and azo compounds, with benzoyl peroxide and azobisisobutyronitrile (AIBN) as more preferred examples.

The initiator solution was prepared by dissolving benzoyl peroxide in methanol. The monomer solution was prepared by mixing acrylic acid (23 mass %), poly(ethylene glycol) methyl ether methacrylate (70 mass %), and 3-(trimethoxysilyl)propyl methacrylate (7 mass %). The initiator solution was heated in a jacketed reactor to 6420 C. with stirring. The monomer solution was added dropwise to the initiator solution. The polymerization solution was stirred and heated at 64° C. for approximately two hours at which time a solution of azobisisobutyronitrile (AIBN) in methanol was added. Stirring and heating at 64° C.; was continued for an additional one hour at which time a second solution of AIBN in methanol was added to the polymerization solution. After stirring and heating at 64° C. for one more hour, a third and final solution of AIBN in methanol was added to the polymerization solution. After about 1.5 hours after addition of the third AIBN solution, a portion of the polymerization solvent was distilled off. The total heating time for the polymerization was approximately 6 hours and 45 minutes. A sample of the adhesive solution was dried in a hood at room temperature. The resultant film was soft and flexible and had absorbent capacity of 0.93 grams of water/grams of dry film. This film had a glass transition temperature of −49° C., as measured by a Differential Scanning Calorimeter (DSC).

Another portion of this adhesive solution was neutralized with sodium hydroxide solution such that 50% of the acrylic acid units were in the sodium salt form. The neutralized binder solution produced a soft, flexible film after drying at room temperature. This film had absorbent capacity of 1.7 grams of liquid/grams of absorbent and a glass transition temperature of −44° C.

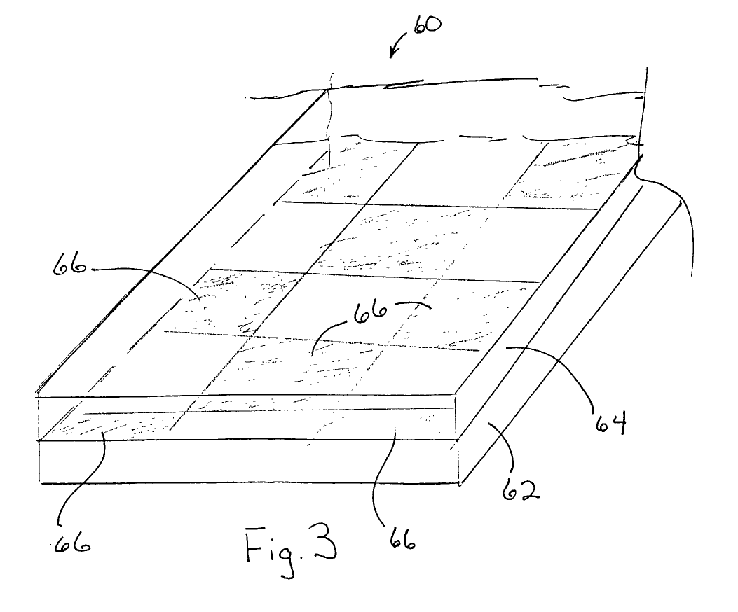

FIGS. 3-5 show the absorbent adhesive in alternative discontinuous patterns including checker board, and a spotted pattern, of circles and diamonds, respectively, although it is not contemplated that such examples will limit all aspects or embodiments of the invention.

As seen in Table 1, a comparison of liquid intake times using a fluid intake flowback evaluation (FIFE) test shows a marked reduction in second intake times. The webs used were pulp webs with 3-5% thermoplastic binder and 40-50% superabsorbent particles. Without limitation, it is contemplated that more generally a pulp web may contain between about 3% and about 15% thermoplastic binder fibers by weight. Samples were prepared in four inch by four inch squares. A control sample, a) in Table 1, with no adhesive between the webs was used. A first sample of a composite according to the invention, b) in Table 1, was prepared with a discontinuous application of a 25% polymer resin solids absorbent adhesive ISOBAM® applied in 3 lines of about ¼ to about ½ inch thickness over the sample. A second sample of a composite according to the invention, c) in Table 1, was prepared with a discontinuous application of a 12.5% polymer resin solids absorbent adhesive applied in 5 lines of about ¼ to about ½ inch thickness over the sample. A comparison sample, d) in Table 1, was prepared with a continuous application of a 12.5% polymer resin solids absorbent adhesive applied between the webs. The percentage of polymer resin solids may vary between about 2.5% and about 50% dependant upon the absorbent adhesive used and the functional application desired. Without limitation, the inventors contemplate that the acrylate copolymer type resins may be used in the higher portions of this range, while the PEO or ISOBAM® polymer resins would more typically be used at up to about 25%.

The samples were then subjected to a serial absorbency test using a FIFE tester as described herein. The FIFE tester has two PLEXIGLAS® plates. The top plate of the FIFE tester includes a cylinder having an inner diameter of 1 inch. The top plate has a circular hole formed in the center thereof. The cylinder extends upwardly substantially perpendicular to the surface of the top plate. The cylinder fits within the circular hole and is secured therein by an adhesive. The adhesive permanently secures the cylinder as an integral part of the top plate and prevents liquid from leaking outwardly onto the top surface of the top plate. A funnel is placed at the top of the cylinder to pour liquid into the test device. The samples were placed between the plates with the cylinder centered over the samples. Fifty cubic centimeters (cc) of 0.9% by weight saline was poured into the cylinder at thirty minute intervals and the time required for the sample to absorb the saline was recorded. Results are reported in seconds.

As can be seen from Table 1, sample c) shows an improvement of intake time for all of first, second and third intakes, over the control and continuous applications of adhesive of samples a) and d), respectively. It is noted that Sample d) did not absorb all 50 cc of liquid within the allotted two minute period. Sample b) shows a marked improvement of intake time for the second intake over the control and continuous applications of adhesive of samples a) and d), respectively. Without wishing to be bound by theory, it is believed that fluid uptake and distribution to all parts of the composite web, including both absorbent webs and the absorbent adhesive application between them, is enhanced by leaving open space within the absorbent adhesive application between the webs.

TABLE 1

| Sample Description | Intake time 1 | Intake time 2 | Intake time 3 |
|---|---|---|---|
| a) no attachment | 46.9 | 53.5 | 53 |
| b) 25% solids, 3 | 50.9 | 24.4 | 44.8 |
| lines | | | |
| c) 12.5% solids, 5 | 43.3 | 21.5 | 60 |
| lines | | | |
| d) 12.5% solids, | 60.9 | 43.8 | NA |
| continuous | | | |

As will be appreciated by those skilled in the art, changes and variations to the invention are considered to be within the ability of those skilled in the art. It will be appreciated that various materials, as well as their amounts, and types, maybe utilized according to the present invention to adapt the invention to a variety of applications while remaining within the spirit of the present invention. Such changes and variations are intended by the inventors to be within the scope of the invention.

We claim:

1. A composite absorbent comprising:
a) a first absorbent layer;
b) a second layer; and
c) a discontinuous layer of adhesive absorbent applied between the first absorbent layer and the second layer so as bind the first absorbent layer and the second layer into a composite absorbent web.

2. The composite absorbent of claim 1 wherein:
the first absorbent layer is a cellulosic layer.

3. The composite absorbent of claim 1 wherein:
the second layer is an absorbent layer.

4. A personal care product comprising the composite absorbent of claim 1.

5. The composite absorbent according to claim 1 wherein the adhesive absorbent comprises between about 2.5% and about 50% polymer resin in an aqueous carrier liquid.

6. The composite absorbent according to claim 5 wherein the adhesive absorbent comprises between about 2.5% and about 25% polymer resin in an aqueous carrier liquid.

7. The composite absorbent according to claim 5 wherein the polymer resin comprises a carboxylated polymer derived from maleic anhydride-isobutylene copolymer.

8. The composite absorbent according to claim 5 wherein the polymer resin comprises an alkoxy silane grafted poly(ethylene oxide).

9. The composite absorbent according to claim 5 wherein the polymer resin comprises acrylate copolymers with moisture curing capability.

10. The composite absorbent according to claim 5 wherein the aqueous liquid consists of water or a mixture of water and miscible organic solvents.

11. The composite absorbent according to claim 1 wherein the discontinuous layer of adhesive absorbent is applied in a distribution pattern comprising stripes.

12. The composite absorbent according to claim 11 wherein the distribution pattern comprises three to five stripes of adhesive per about four inches of material.

13. The composite absorbent according to claim 12 wherein the stripes are between about one quarter and about one half inch in width.

14. The composite absorbent according to claim 11 wherein the stripes are about 1 cm in width and spaced about 2.5 cm center to center.

15. The composite absorbent according to claim 1 wherein the first absorbent layer is an airlaid absorbent layer.

16. The composite absorbent according to claim 15 wherein the airlaid absorbent layer contains pulp, superabsorbent material, and 3-15% by weight thermoplastic binder fibers.

17. The composite absorbent according to claim 1 wherein the first absorbent layer is a foam layer.

18. The composite absorbent according to claim 1 wherein the first absorbent layer is a nonwoven layer.

19. The composite absorbent according to claim 1 wherein the first absorbent layer and the second layer are densified layers.

20. The composite absorbent according to claim 1 further comprising about 1 gsm to about 200 gsm of the adhesive absorbent.

21. The composite absorbent according to claim 1 further comprising about 2 gsm to about 150 gsm of the adhesive absorbent.

22. The composite absorbent according to claim 1 further comprising about 5 gsm to about 100 gsm of the adhesive absorbent.

23. The composite absorbent according to claim 1 wherein the area of adhesive absorbent coverage is between about 5% and about 95% of the area between the first absorbent layer and the second layer.

24. The composite absorbent according to claim 1 wherein the area of adhesive absorbent coverage is between about 10% and about 80% of the area between the first absorbent layer and the second layer.

25. The composite absorbent according to claim 1 wherein the area of adhesive absorbent coverage is between about 20% and about 60% of the area between the first absorbent layer and the second layer.

26. An absorbent article comprising:
a) a liquid permeable liner layer;
b) a liquid impermeable back sheet layer;
c) an absorbent structure located between the liquid permeable liner layer and the liquid impermeable back sheet layer, the absorbent structure comprising:
i) a first absorbent layer suitable for placement in an absorbent article;
ii) a second layer suitable for placement in an absorbent article; and
iii) a discontinuous layer of adhesive absorbent applied between the first absorbent layer and the second layer so as bind the first absorbent layer and the second layer into a composite absorbent.

27. The absorbent article according to claim 26 wherein the first absorbent layer is primarily a liquid retention layer.

28. The absorbent article according to claim 26 wherein the first absorbent layer and the second layer are primarily liquid retention layers.

29. The absorbent article according to claim 26 wherein the first absorbent layer is primarily a liquid acquisition and distribution layer.

30. The absorbent article according to claim 26 wherein the first absorbent layer is an airlaid absorbent layer.

31. The absorbent article according to claim 30 wherein the airlaid absorbent layer contains pulp, superabsorbent material, and 3-15% by weight thermoplastic binder fibers.

32. The absorbent article according to claim 26 wherein the first absorbent layer is a foam layer.

33. The absorbent article according to claim 26 wherein the first absorbent layer is a nonwoven layer.

34. The absorbent article according to claim 26 wherein the first absorbent layer and the second layer are densified layers.

35. The absorbent article according to claim 26 further comprising about 1 gsm to about 200 gsm of the adhesive absorbent.

36. The absorbent article according to claim 26 further comprising about 2 gsm to about 150 gsm of the adhesive absorbent.

37. The absorbent article according to claim 26 further comprising about 5 gsm to about 100 gsm of the adhesive absorbent.

38. The absorbent article according to claim 26 wherein the area of adhesive absorbent coverage is between about 5% and about 95% of the area between the first absorbent layer and the second layer.

39. The absorbent article according to claim 26 wherein the area of adhesive absorbent coverage is between about 10% and about 80% of the area between the first absorbent layer and the second layer.

40. The absorbent article according to claim 26 wherein the area of adhesive absorbent coverage is between about 20% and about 60% of the area between the first absorbent layer and the second layer.

41. A method of making a composite absorbent comprising:
a) providing a first web, the first web being primarily a liquid retention layer;
b) applying an adhesive in a discontinuous distribution onto the first web;
c) contacting the first web with a second web with the adhesive between the first web and the second web;
d) treating the contacted first web and second web and the adhesive to perform at least one of a removal of excess water or a crosslinking of the adhesive so as to make the treated adhesive absorbent.

42. The method of making a composite absorbent of claim 41 wherein the adhesive is applied in a liquid solution.

43. The method of making a composite absorbent of claim 41 wherein the adhesive is applied in a dry state.

44. The method of making a composite absorbent of claim 41 wherein the step of treating includes heating.

45. The method of making a composite absorbent of claim 41 wherein the step of treating includes exposure to moisture.

46. The method of making a composite absorbent of claim 41 wherein the step of contacting includes compressing the first web and the second web together.
